(19) United States
(12) Patent Application Publication
(10) Pub. No.: US 2009/0319036 A1
(43) Pub. Date: Dec. 24, 2009

(54) Medical Devices Made From Polymers With End Group Modification For Improved Thermal Stability

(76) Inventors: Yunbing Wang, Sunnyvale, CA (US); Stephen D. Pacetti, San Jose, CA (US)

Correspondence Address: SQUIRE, SANDERS & DEMPSEY LLP, 1 MARITIME PLAZA, SUITE 300, SAN FRANCISCO, CA, 94111, US

(21) Appl. No.: 12/142,562

(22) Filed: Jun. 19, 2008

(51) Int. Cl. A61F 2/82; B29D 22/00

(52) U.S. Cl. 623/1.49; 264/563; 264/564

(57) ABSTRACT

Implantable medical devices and methods of forming thereof made from polymers with end groups providing improved thermal stability are disclosed. Implantable medical devices made from such polymers including stabilizing agents are additionally disclosed.

### BACKGROUND OF THE INVENTION

1. Field of the Invention

This invention relates to implantable medical devices, such as stents, made from polymers with improved thermal stability.

2. Description of the State of the Art

This invention relates to various kinds of implantable medical devices including structures made from polymers. Such implantable medical devices include, but are not limited to, radially expandable prostheses, such as stents and stent grafts, catheters, and pacemaker leads.

Radially expandable endoprostheses are adapted to be implanted in a bodily lumen. An “endoprosthesis” refers to an artificial device that is placed inside the body. A “lumen” refers to a cavity of a tubular organ such as a blood vessel. Stents are generally cylindrically shaped devices, which function to hold open and sometimes expand a segment of a blood vessel or other anatomical lumen such as urinary tracts and bile ducts. Stents are often used in the treatment of atherosclerotic stenosis in blood vessels. “Stenosis” refers to a narrowing or constriction of the diameter of a bodily passage or orifice. In such treatments, stents reinforce body vessels and prevent restenosis following angioplasty in the vascular system. “Restenosis” refers to the reoccurrence of stenosis in a blood vessel or heart valve after it has been treated (as by balloon angioplasty, stenting, or valvuloplasty) with apparent success.

The treatment of a diseased site or lesion with a stent involves both delivery and deployment of the stent. “Delivery” refers to introducing and transporting the stent through a bodily lumen to a region, such as a lesion, in a vessel that requires treatment.

“Deployment” corresponds to the expanding of the stent within the lumen at the treatment region. Delivery and deployment of a stent are accomplished by positioning the stent about one end of a catheter, inserting the end of the catheter through the skin into a bodily lumen, advancing the catheter in the bodily lumen to a desired treatment location, expanding the stent at the treatment location, and removing the catheter from the lumen.

In the case of a balloon expandable stent, the stent is mounted about a balloon disposed on the catheter. Mounting the stent typically involves compressing or crimping the stent onto the balloon. The stent is then expanded by inflating the balloon. The balloon may then be deflated and the catheter withdrawn. In the case of a self-expanding stent, the stent may be secured to the catheter via a constraining member such as a retractable sheath or a sock. When the stent is in a desired bodily location, the sheath may be withdrawn which allows the stent to self-expand.

The stent must be able to satisfy a number of requirements such as the radial strength necessary to withstand the structural loads, namely radial compressive forces, imposed on the stent as it supports the walls of a vessel. Once expanded, the stent must adequately maintain its size and shape throughout its service life despite the various forces that may come to bear on it, including the cyclic loading induced by the beating heart. For example, a radially directed force may tend to cause a stent to recoil inward. In addition, the stent must possess sufficient flexibility to allow for crimping, expansion, and cyclic loading. Finally, the stent must be biocompatible so as not to trigger any adverse vascular responses.

The structure of a stent is typically composed of scaffolding that includes a pattern or network of interconnecting structural elements often referred to in the art as struts or bar arms. The scaffolding can be formed from wires, tubes, or sheets of material rolled into a cylindrical shape. The scaffolding is designed so that the stent can be radially compressed (to allow crimping) and radially expanded (to allow deployment).

Additionally, a medicated stent may be fabricated by coating the surface of either a metallic or polymeric scaffolding with a polymeric carrier that includes an active or bioactive agent or drug. Polymeric scaffolding may also serve as a carrier of an active agent or drug.

Furthermore, it may be desirable for implantable medical devices, such as stents, to be biodegradable. In many treatment applications, the presence of a stent in a body may be necessary for a limited period of time until its intended function of, for example, maintaining vascular patency and/or drug delivery is accomplished. Therefore, stents fabricated from biodegradable, bioabsorbable, and/or bioerodable bioabsorbable polymers can be configured to partially or completely erode away after the clinical need for them has ended.

### SUMMARY OF THE INVENTION

Various embodiments of the present invention include a stent comprising a portion formed of a biodegradable polyester, wherein the polyester has selected end groups that provide greater thermal stability and reduced chemical degradation of the polyester compared to that with hydroxyl end groups.

Further embodiments of the present invention include a method of fabricating a stent comprising: processing a biodegradable polyester to form a stent, the processing subjects the polyester to conditions that cause chemical degradation of the biodegradable polyester, wherein the polyester has selected end groups that provide greater thermal stability and reduced chemical degradation to the polyester during the processing than provided by hydroxyl end groups.

Additional embodiments of the present invention include a method of fabricating a stent comprising: forming a tube made from a biodegradable polyester using melt extrusion; and laser machining a stent pattern in the tube to form a stent, wherein the polyester has selected end groups that provide greater thermal stability to the polyester than provided by hydroxyl end groups during the forming and machining steps.

### BRIEF DESCRIPTION OF THE DRAWINGS

FIG. 1 depicts a stent.

FIG. 2 depicts results of thermagravimetric analysis of a PLLA star polymer for different end groups.

FIG. 3 depicts a reaction showing end group modification of a linear or a star PLLA polymer with —OH end groups to —CL and —NH₂ end groups.

FIG. 4 depicts a reaction showing end group modification of a linear or a star PLLA polymer with —OH end groups to —COOH end groups.

FIG. 5 depicts a reaction showing end group modification of a linear or a star PLLA polymer with —OH end groups to acetyl end groups.

FIG. 6 depicts a reaction of linear or star PLLA-OH with diazomethane to form PLLA-O—CH₃.

FIG. 7 depicts a reaction of linear or star PLLA-OH with a monoisocyanate to form a polyurethane.

FIG. 8 depicts a reaction of an —OH terminated biodegradable polyester to form a biodegradable polyester with tertiary amine end groups.

FIG. 9 depicts a reaction of an —OH terminated biodegradable polyester to form a biodegradable polyester with carbonate end groups.

### DETAILED DESCRIPTION OF THE INVENTION

Embodiments of the present invention relate to implantable medical devices and methods of fabricating thereof made from polymers having end groups that provide improved thermal stability and reduced chemical degradation of the polymer during one or more processing steps of a fabrication process. Such processing steps include exposing the polymer to conditions such as elevated temperatures and radiation that can cause chemical degradation to a polymer. The methods and devices described herein are generally applicable to any implantable medical device. In particular, the methods can be applied to tubular implantable medical devices such as self-expandable stents, balloon-expandable stents, stent-grafts, and pacemaker leads. Additionally, the invention applies to devices made partially or completely of the modified polymer. For example, a device body such as a stent scaffolding can be made from the modified polymer. Additionally, a coating over a device body can also be made from a modified polymer.

The embodiments are particularly relevant, for reasons discussed below, to implantable medical devices, such as stents, having a polymeric substrate or scaffolding, a polymer-based coating, a drug-delivery coating, or a combination thereof.

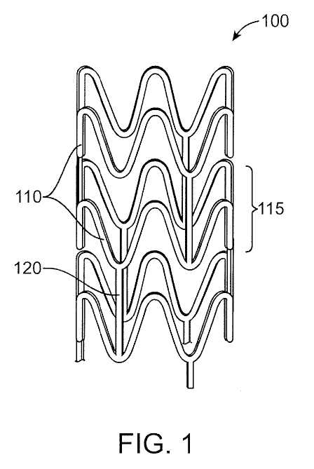

An exemplary structure of a stent is shown in FIG. 1. FIG. 1 depicts a stent 100 which is made up of struts 110. Stent 100 has interconnected cylindrical rings 115 connected by linking struts or links 120. The embodiments disclosed herein are not limited to fabricating stents or to the stent pattern illustrated in FIG. 1. The embodiments are easily applicable to other stent patterns and other devices. The variations in the structure of patterns are virtually unlimited. A stent such as stent 100 may be fabricated from a tube by forming a pattern in the tube with a technique such as laser cutting or chemical etching.

Furthermore, stents and other implantable medical devices have been designed for the localized delivery of a therapeutic agent. A medicated stent may be constructed by coating the device or substrate with a coating material containing a therapeutic agent. The substrate of the device may also contain a therapeutic agent.

In embodiments of the present invention, an implantable medical device can be made partially or completely from a biodegradable, bioabsorbable, or biostable polymer. A polymer for use in fabricating an implantable medical device can be biostable, bioabsorbable, biodegradable or bioerodable. Biostable refers to polymers that are not biodegradable. The terms biodegradable, bioabsorbable, and bioerodable are used interchangeably and refer to polymers that are capable of being completely degraded and/or eroded when exposed to bodily fluids such as blood and can be gradually resorbed, absorbed, and/or eliminated by the body. The processes of breaking down and absorption of the polymer can be caused by, for example, hydrolysis, enzymolysis, oxidation, and metabolic processes. In some treatment situations, a degradation time of less than 2 years may be desirable, for example, between 6 and 14 months, or more narrowly, between 8 and 12 months. Degradation time refers to the time for complete erosion of a device from an implant site or in vitro.

The present invention is applicable to biodegradable polyesters including, but not limited to, poly(L-lactide) (PLLA), poly(DL-lactide) (PDLLA), polyglycolide (PGA), xzpoly(glycolide-co-lactide) (PLGA), poly(L-lactide-co-caprolactone) (PLLA-co-PCL), poly(L-lactide-co-trymethylene carbonate) (PLLA-co-PTMC), and poly(L-lactide-co-DL-lactide) (PLLA-co-PDLLA). In general, the present invention is applicable to any biodegradable polyester made by ring-opening polymerization. It also applies to biodegradable polycarbonates and polydepsipeptides made by ring opening polymerization.

In general, the chemical degradation of a polymer, such as a biodegradable polyester, can arise from exposure to heat, light and other types of radiation, oxygen, and moisture. The presence of acids, bases, or metallic ions can catalyze the degradative process. Chemical degradation results in a decrease in molecular weight of polymer due to chemical reactions within a polymer chain, between polymer chains and other species, and between polymer chains. These chemical reactions, thus, can result in the reduction in molecular weight of the polymer or molecular weight degradation.

The chemical degradation of polymers can arise from several different chemical reaction mechanisms. In one mechanism, moisture can react with polymer chains by hydrolysis, resulting in chain scission and a reduction in molecular weight. In other mechanisms, heat, light and other types of radiation cause chain scission by free radical reactions and non-free radical reactions. Oxygen can accelerate and propagate the free radical reactions. Free radical formation results in chain scission, resulting in the formation of a series of byproducts, such as monomers (e.g., lactide monomers from PLLA), cyclic oligomers, and shorter polymer chains.

Exemplary nonradical mechanisms of chemical degradation includes initiation of chain scission by end groups of the polymer chains. This mechanism is referred to as a “back-biting” interchange reaction involving hydroxyl chain end groups. The highly reactive hydroxyl end groups of biodegradable polyesters can react with ester moieties along the polymer chains. The back-biting reaction causes an “unzipping” depolymerization starting with the hydroxyl ends groups to form monomers and cyclic oligomers. This is essentially the reverse of the polymerization process.

In addition, chemical degradation may be initiated or facilitated by the presence of residual metal, such as from a catalyst used in the synthesis of the polymer. Additionally, transesterification reactions can result in chain scission and reduction in molecular weight.

Poly(L-lactide), for example, typically has at least one hydroxyl end group and has the general formula: R—[OCH(CH3)CO]n-OH, which will be abbreviated as: PLLA-OH. Poly(L-lactide) is subject to thermal degradation at elevated temperatures, with significant degradation (measured as weight loss) starting at about 140° C. and increasing at higher temperatures. The polymer is subject to chemical degradation by both free radical and non-free radical mechanisms that result in random chain scission which generates byproducts such as oligomers and lactide monomers. To explain the presence of lactide at higher temperatures, it has been postulated that there is an equilibrium between the lactide monomer and the polymer chain. In addition to lactide, the degradation products also include aldehydes, and other cyclic oligomers.

It has been proposed that for PLLA-OH, the main thermal chemical degradation process is the backbiting ester interchange reaction involving OH end groups. McNeill et al., Polym. Degrad. Stab. 1985; 11:309-26. Three back-biting degradation reactions have been proposed. In each of these reactions, the —OH end structure is regenerated which allows the degradation reactions to continue.

Fabricating polymer stents can involve processing steps that expose the polymer to high temperatures and other conditions such as radiation that can result in chemical degradation. In particular, melt processing techniques such as extrusion and injection molding can result in a decrease in molecular weight and formation of lower molecular weight species. The decrease in molecular weight can adversely affect mechanical properties and other properties of the polymer such as degradation behavior and drug release properties.

In general, it is desirable to reduce or eliminate molecular weight reduction as a polymer is processed from an initial raw material to the final product so that the final product will have acceptable properties, such as mechanical properties and in vivo degradation properties. Also, reducing or preventing chemical degradation during processing will make a processed polymer less sensitive to variations in processing conditions. As a result, the properties of processed polymers will be more consistent from batch to batch. For example, an extruded polymer will be less sensitive to variations in temperature during extrusion.

Both mechanical properties and in vivo degradation behavior are sensitive to chemical degradation of a polymer. In general, mechanical properties, such as strength and toughness, tend to decrease as molecular weight decreases. The in vivo degradation rate or degradation time of a polymer also tends to decrease with a decrease in molecular weight. For instance, it has been observed that PLLA processed by melt extrusion, laser machining, and radiation sterilization exhibits a shorter degradation time than the raw material originally fed to the extrusion process. For example, a PLLA not subjected to melt processing typically has a degradation time of 2-3 years. A melt processed PLLA can have a degradation time of less than a year. It is believed this decrease in degradation time is due to the chemical degradation of the polymer during processing. In particular, the reduced degradation time may be caused, at least in part, by the presence of monomers and other low molecular weight species that are byproducts of polymer chemical degradation during processing.

In particular, the fabrication of a polymeric implantable medical device, such as a stent, can include several processing steps, such as:

(1) forming a polymeric construct, such a tube, by melt processing, such as extrusion;

(2) radially deforming the formed tube;

(3) forming a stent body from a tube by laser machining a stent pattern in the deformed tube;

(4) application of a therapeutic coating on the stent body;

(5) crimping the stent over catheter balloon;

(6) radiation sterilization of the stent

The polymer is particularly sensitive to chemical degradation or decomposition during extrusion, laser machining, and sterilization steps. Additionally, chemical degradation or decomposition can occur during one or more of the other processing steps. The degree of chemical degradation and molecular weight degradation can depend upon the temperature range the polymer is exposed to during processing and the duration of exposure.

A polymer tube can be formed through melt processing methods such as extrusion and injection molding. In extrusion, a polymer melt is conveyed through an extruder barrel to an exit port. The polymer is fed to an extruder barrel near its proximal end in a solid form, for example, as a pellet from a hopper. The polymer in the extruder barrel is heated to temperatures near or above the melting temperature (Tm) of the polymer and exposed to shear forces and pressures that are generally far above ambient. Since the viscosity decreases with temperature, the temperature is at a level that allows a desired flow rate of polymer through the extruder. Exemplary processing temperatures in an extruder can be at or about Tm of the polymer, up to 10° C. above, 10-30° C. above, 30-50° C. above, or more than 50° C. above the Tm of the polymer. The Tm's of some exemplary polymers are given in Table 1. The extrusion process may result in a decrease in molecular weight, for example, of up to 20%, 20%-40%, 40%-60%, or greater than 60% of the initial molecular weight. In exemplary embodiments, PLLA can be extruded at a temperature of at least about 180° C., 180-210° C., 210-230° C., or greater than 230° C.

TABLE 1
Melting temperatures and glass transition
temperatures of exemplary polymers.

| Polymer | Melting Point (° C.)¹ | Glass Transition Temp (° C.)¹ |
|---|---|---|
| PGA | 225-230¹ | 35-40 |
| PLLA | 173-178¹ | 60-65 |
| PDLLA | Amorphous | 55-60 |
| PCL | 58-63¹ | (−65)-(−60) |
| | 60² | |
| PDO | N/A | (−10)-0 |
| 85/15 PLGA | Amorphous | 50-55¹ |
| 75/25 PLGA | Amorphous | 50-55¹ |
| 65/35 PLGA | Amorphous | 45-50¹ |
| 50/50 PLGA | Amorphous | 45-50¹ |

¹Plastics and Biomaterials Magazine, March 1998.
²Science, Vol. 297 p. 803 (2002)

The polymer melt exits the distal end of the extruder barrel into a die. The die imparts a cylindrical shape to the polymer melt exiting the die, which is cooled to form a tube. Representative examples of extruders for use with the present invention may include single screw extruders, intermeshing co-rotating and counter-rotating twin-screw extruders, and other multiple screw masticating extruders.

Radial expansion of the polymer tube may be performed to increase the radial strength of the tube, prior to cutting a stent pattern. Generally, deformation of a polymer construct can result in an increase in strength in the deformed construct along the direction of deformation. Therefore, radial expansion or deformation can result in an increase in radial strength of a polymer tube. The increase in strength is believed to arise from molecular orientation induced along the direction of deformation which can increase the strength and modulus along the direction of deformation.

However, since the radial expansion process is preferably performed at an elevated temperature, it may result in chemical degradation of the polymer of the tube. The tube can also be axially deformed to increase strength in the axial direction. The tube polymer is generally heated to a temperature above a glass transition temperature (Tg) of the polymer, but less than the Tm of the polymer. The Tg's of exemplary polymers are given in Table 1. An exemplary polymer tube can be heated to a temperature during blow molding to at least 5° C. above Tg, up to 20° C. above Tg, 20-60° C. above Tg, or 60° C. above Tg to 10° C. below Tm. For example, a PLLA tube can be heated to a temperature during blow molding of at least about 65° C., 65-80° C., 80-120° C., or 120-163° C.

As an illustration, blow molding can be used to radially deform a polymer tube. The polymeric tube is placed in a mold, and deformed in the radial direction through an increase in the pressure inside the tube by blowing a gas into the tube. The increased pressure expands the tube and the mold limits the radial deformation of the polymeric tube to the inside diameter of mold.

During the blow molding, the polymer tube may be heated by a heated gas or fluid directed on the mold, by a heated mold, or by heating the gas blown into the tube. After the tube has been blow molded to a particular diameter, the tube can be maintained under the elevated pressure and temperature for a period of time. The period of time may be between about one minute and about one hour, or more narrowly, between about two minutes and about ten minutes. This is referred to as “heat setting.” Heating setting can be performed in the exemplary temperature ranges provided above for blow molding.

Laser cutting or machining can be used to cut a stent pattern in a polymer tube. Laser cutting can result in chemical degradation in all or a part of the tube due to heat and radiation exposure from the laser beam. In particular, chemical degradation can be most significant in selected or localized regions called a heat affected zone (HAZ). A HAZ refers to portions of a target substrate adjacent to removed substrate material which although not removed, are still exposed to energy from the laser beam, either directly or indirectly. Direct exposure may be due to exposure from a section of the beam with an intensity that is not great enough to remove substrate material. A substrate can also be exposed to energy indirectly due to thermal conduction and radiation scattering. The increased temperature in a HAZ can lead to chemical degradation of the polymer.

In some embodiments, the extent of a HAZ may be mitigated by the use of an ultrashort-pulse laser. “Ultrashort-pulse lasers” refer to lasers having pulses with durations shorter than about a picosecond (=10⁻²), and includes both picosecond and femtosecond (=10⁻¹⁵) lasers. Ultrashort pulse lasers remove material by means of a nonthermal mechanism which results in reduced energy transfer into a substrate. Other embodiments include laser machining a stent pattern with a conventional continuous wave or long-pulse laser (nanosecond (10⁻⁹) laser) which has significantly longer pulses than utlrashort pulse lasers. There is a larger HAZ for a continuous or long-pulse laser as compared to an ultrashort pulse laser, and therefore, the extent of polymer degradation is higher.

A coating step may potentially result in chemical degradation of the stent body or scaffolding and a polymeric coating due to exposure to moisture or heating during a drying step described below. The stent body formed from cutting the stent pattern into the polymeric tube may be coated. The coating may be polymeric or non-polymeric and may include an active agent. A polymeric coating can be formed by applying a coating composition containing a polymer dissolved in a solvent. An active agent can be mixed or dispersed in the solvent as well. The coating composition is generally applied at ambient temperature (20-30° C.). The coating is formed through removal of the solvent by a drying step that can include applying heat to the stent. The drying step can include blowing a heated gas on the stent, for example, for 10 to 45 seconds, at 35-45° C. or greater than 45° C. The application and drying steps can be repeated several times to achieve a desired mass of polymer coating, active agent, or both.

When coating application is completed, residual solvent can be removed by heating the coated stent to temperatures of 40-65° C., or greater than 65° C. For example, the coated stent can be placed in a vacuum oven between 30-180 min. Exposure to the elevated temperatures during the drying processes may lead to chemical degradation of the polymer of the coating, the scaffolding, or both.

Mounting the stent on a support element for delivery can also result in chemical degradation of the polymer in the stent. A stent can be mounted on a support element, such as a catheter balloon, by crimping the stent over the support element. In some embodiments, a stent can be heated to a temperature above ambient during the crimping process. The crimping of a polymeric stent can be facilitated and mechanical behavior of the stent after crimping can be improved by such heating. For example, heating a stent during crimping can reduce or eliminate recoil of a crimped stent prior to delivery in a lumen. In exemplary embodiments, a stent can be crimped in a temperature in a range up to the Tg or above the Tg of the scaffolding polymer, for example, between −5% to 5% the Tg of the polymer of the scaffolding polymer or greater than 5% above the Tg of the scaffolding polymer. For example, for a PLLA stent, crimping may be performed at a temperature range of 30-60° C. or greater than 60° C. for a duration ranging from about 60 seconds to about 5 minutes.

Sterilization of a stent can also cause chemical degradation of polymer in an implantable medical device, such as a stent. Typically, a stent is sterilized after crimping and packaging the crimped stent. Ethylene oxide sterilization, or irradiation, either gamma irradiation or electron beam irradiation (e-beam irradiation), are typically used for terminal sterilization of medical devices. For ethylene oxide sterilization, the medical device is exposed to liquid or gas ethylene oxide that sterilizes through an alkylation reaction that prevents organisms from reproducing. Ethylene oxide penetrates the device, and then the device is aerated to assure very low residual levels of ethylene oxide because it is highly toxic. Thus, the ethylene oxide sterilization is often performed at elevated temperatures to speed up the process. Chemical degradation of the polymer can occur due to the ethylene oxide interactions with the polymer, moisture exposure, and to the elevated temperatures.

Radiation sterilization can also cause chemical degradation to the polymer in a stent. It is known that radiation can alter the properties of polymers. High-energy radiation, such as electron beams (e-beam) and gamma radiation, tends to produce ionization and excitation in polymer molecules. These energy-rich species undergo dissociation, subtraction, and addition reactions in a sequence leading to chemical degradation. The degradation can occur during, immediately after, or even days, weeks, or months after exposure to radiation which often results in physical and chemical cross-linking or chain scission. Resultant physical changes can include embrittlement, discoloration, odor generation, stiffening, and softening, among others.

In particular, the deterioration of the performance of polymers due to e-beam radiation sterilization has been associated with free radical formation during radiation exposure and by reaction with other parts of the polymer chains. The reaction is dependent on e-beam dose and temperature. Additionally, exposure to radiation, such as e-beam, can cause a rise in temperature of an irradiated polymer sample. The rise in temperature is dependent on the level of exposure. In particular, the effect of radiation on mechanical properties becomes more profound as the temperature approaches and surpasses the Tg of the polymer. The deterioration of mechanical properties may result from the effect of the temperature on polymer morphology, but also from increased degradation resulting in a decrease in molecular weight.

The degree of chemical degradation can be reduced by irradiating the stent before, during, or after the stent is cooled to a temperature below ambient temperature. As an example, without limitation, a stent can be sterilized while the stent is at a temperature of less than −30° C., −30° C. to 0° C., or 0° C. to 25° C. The sterilization may occur in multiple passes through the electron beam. In other embodiments, the stent can be at ambient temperature when it is irradiated.

As outlined above, the manufacturing process of a stent exposes the stent to conditions such as high temperature and radiation that can chemically degrade the stent polymer. In addition, residual catalysts in the polymer raw material and other metals, such as from processing equipment, may catalyze degradation reactions. Exposure to shear stress during extrusion can also cause chemical degradation. Thus, there are a number of sources of potential chemical degradation of a stent polymer during processing.

Polymer molecular weight may significantly decrease during the processing steps of stent manufacture. A non-limiting example is fabrication of a stent having a stent scaffolding made from PLLA polymer. The stent manufacturing process involves extruding a polymer tube, radially expanding the polymer tube, laser cutting a stent pattern into the tube to form a stent, crimping the stent onto a balloon catheter, and sterilizing the crimped stent with e-beam radiation. In the absence of thermal stabilizers in the polymer or end group modification, the process can cause a decrease of the weight average molecular weight (Mw) from about 550 kg/mol to about 190 kg/mol. Extrusion of the polymer tube can result in a decrease to about 380 kg/mol from the initial 550 kg/mol. The molecular weight is further decreased to about 280 kg/mol after radial expansion and laser cutting. After sterilization by electron beam irradiation (25 KGy), the molecular weight (weight average) is about 190 kg/mol.

Various embodiments of the present invention include modifying end groups of a biodegradable polyester to provide greater thermal stability and reduced chemical degradation of the polymer upon exposure to conditions that can cause chemical degradation or decomposition of the polymer. The conditions include elevated temperatures and radiation that can cause such degradation. In particular, the conditions include the processing conditions during the stent processing steps described above.

In certain embodiments, the hydroxyl end groups of biodegradable polyesters are replaced with selected functional groups that improve the thermal stability and reduce chemical degradation of the polymer during processing. Such selected functional groups have no or a reduced tendency to initiate chain scission reactions on the polymer chain. Therefore, the chemical degradation of the polymer due to back-biting reactions can be reduced. As a result, the degree of decomposition at a given temperature is reduced by the end group modification. It is expected that the degree of molecular weight decomposition due to each of the steps can be reduced up to 10%, 10-50%, 50-80%, or greater than 80%.

The degree of thermal stabilization through end group modification depends on the relative importance of back-biting reactions on decomposition of the biodegradable polyester polymer. The more significant the backbiting reaction is to chemical degradation, the more the end group modification improves the thermal stability of the polymer.

Further embodiments of the present invention include processing the end group modified biodegradable polyester to form an implantable medical device such as a stent. The processing includes steps, described above, including extrusion, radial expansion, laser machining, coating, and sterilization. The selected functional groups reduce molecular weight degradation that are due to conditions in these processing steps such as elevated temperature and radiation. The end group modification can result in the polymer having a higher molecular weight at the end of each processing step as compared to an unmodified polymer.

In these embodiments, some or all of the hydroxyl end groups of the polymer can be replaced by the selected end groups. In exemplary embodiments, less than 10%, 10-30%, 30-70%, or greater than 70% of hydroxyl groups can be replaced by the selected end group. Additionally, the polymer chains of the polymer can be linear, star, branched, or dendritic.

Embodiments of the present invention include several types of end group modification. Such embodiments include end group modification of biodegradable polymers involving replacement of a hydroxyl group with functionalities that provide greater thermal stability:

(1) Replace —OH end group with —Cl or —NH₂ group

(2) Replace —OH end group with —COOH group

(3) Replace —OH end group with acetyl group

(4) Replace —OH end group with ether functionality

(5) Replace —OH end group with urethane

(6) Replace —OH end group with tertiary amine

(7) Replace —OH end group with carbonate

### (1) —Cl or —NH₂ Group

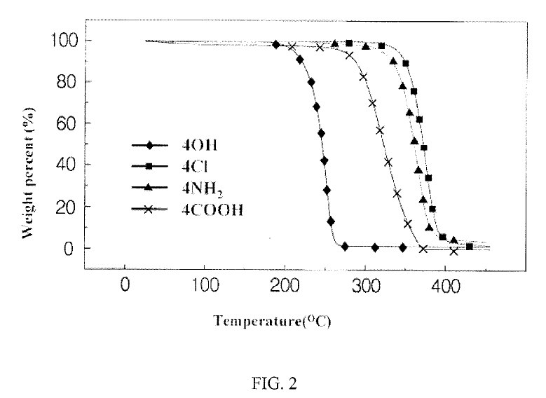

The effect of end group modification on the thermal stability has been demonstrated. For example, in PLLA-OH, replacement of —OH end group with —Cl, —NH₂, or —COOH end groups has been shown to result in greater thermal stability as reflected by increased decomposition temperatures. Lee et al., J. Polymer Sci.: Part A: Polymer Chemistry, Vol. 39, 973-985 (2001). FIG. 2 depicts results of thermagravimetric analysis of PLLA star polymers with different end groups. Ibid. FIG. 2 shows the weight percent of PLLA remaining as a function of the temperature. The PLLA polymers with the —Cl, —NH₂, or —COOH end groups each decompose or degrade at higher temperatures than the PLLA with —OH end groups.

End group modification of an —OH terminated polymer to have —Cl or —NH₂ terminated groups can be performed on a variety of polymers, such as biodegradable polyesters. A non-limiting example includes modifying linear or star PLLA to have —Cl or —NH₂ groups. FIG. 3 depicts a reaction showing end group modification of a linear or a star PLLA polymer with —OH end groups. The linear or star PLLA-OH reacts with thionyl chloride to form PLLA-CL. The PLLA-CL is then reacted with sodium azide, followed by reduction, to form PLLA-NH₂.

In an exemplary embodiment, 200 g —OH terminated PLLA is dissolved in 1 L toluene with 20 g pyridine and then 20 ml thionyl chloride is added drop-wise for 20 min at 100° C. The modified polymer, PLLA-Cl, is precipitated into an excess, e.g., 4 L, of methanol, filtered and dried in vacuum.

NH₂ terminated PLLA can be made by adding sodium azide to a solution of —Cl terminated PLLA in N,N-dimethylformamide (DMF). A solution of PLLA-Cl is stirred while at about 80° C. for 2 h. The polymer solution is then poured in an excess of methanol, filtered, and dried under vacuum to form —N₃ terminated PLLA. The PLLA-N₃ is dissolved in chloroform, then 10% palladium on activated carbon is added. The mixture is then hydrogenated in a hydrogenation apparatus for 6 hours. The catalyst is removed with a filter, e.g., a 0.45 mm pore membrane filter. The polymer is precipitated by pouring the solution into an excess of methanol, filtered, and dried under vacuum.

### (2) —COOH Group

Modifying —OH end groups of a biodegradable polyester to carboxylic acid (—COOH) groups increases the thermal stability and can increase the degradation rate of the modified biodegradable polymer in an aqueous environment. Specifically, the carboxylic acid end group can increase the rate of hydrolysis reactions, and thus, the degradation rate of the polymer. Therefore, the end group modification can increase the degradation rate of an implantable medical device, such as a stent, in vivo that is made from the modified polymer.

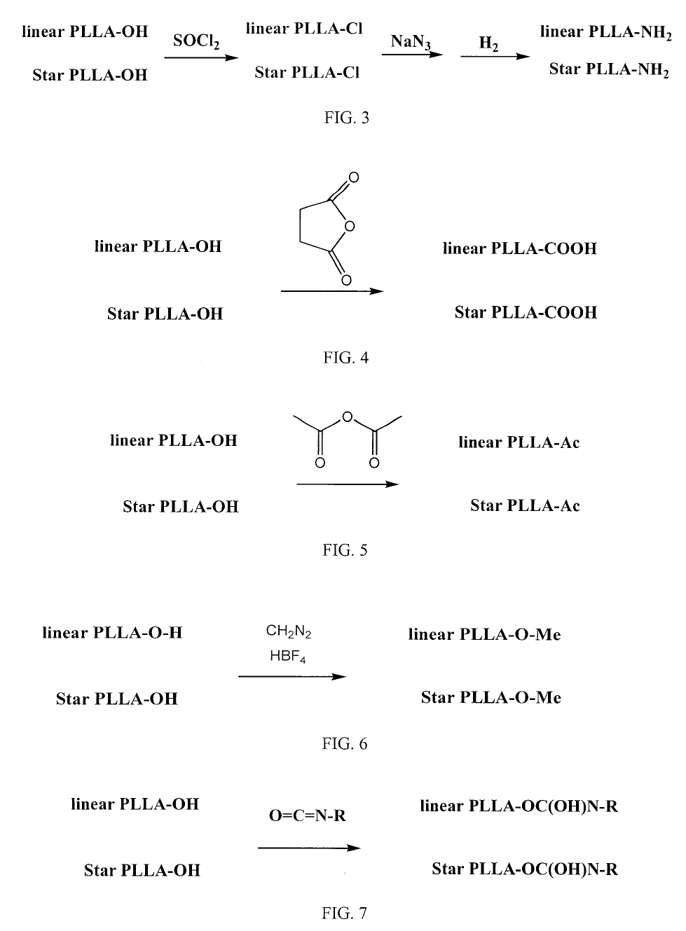

FIG. 4 depicts a reaction showing end group modification of a linear or a star PLLA polymer with —OH end groups. The PLLA-OH is in a solution of 1,4 dioxanone with succinic anhydride, 4-dimethyl aminopyridine (DMAP), and triethylamine (TEA). The solution is stirred (e.g., 12 hours) to facilitate the reaction. The PLLA-COOH polymer is precipitated in an excess of methanol. A final product is obtained by filtering the precipitate and drying in vacuum.

### (3) Acetyl Group

Modification of an —OH terminated biodegradable polyester to have acetyl groups (—COCH₃) is also expected to increase the thermal stability of biodegradable polyesters. Acetylation has been shown to increase the thermal stability of —OH terminated PLLA. McNeill et al., Polym. Degrad. Stab. 1985; 11:309-26. This can be due in part to the reducing or prevention back-biting reactions.

In addition, it is believed that residual metals, such as Sn and Al, from the polymerization catalysts can accelerate chemical degradation of polymers. The presence of such metals can greatly decrease the thermal stability of a biodegradable polyester during stent processing. Acetyl ends groups can reduce the effect of such residual metals on chemical degradation of the polymer during stent processing.

FIG. 5 shows an acetylation reaction of linear or star PLLA-OH. The PLLA-OH reacts with acetic anhydride to form acetylated PLLA. In an exemplary synthesis scheme, 200 g PLLA-OH is dissolved in 1000 ml of chloroform. 20 g of acetic anhydride is then added into the solution. The solution is heated at 60° C. for 4 hours while being stirred in an N₂ atmosphere. Acetylated PLLA is then precipitated into 4 L methanol, filtered, and dried in a vacuum oven, e.g., at 90° C. for 48 hours.

### (4) Ether Functionality Group

Other embodiments of modifying end groups of biodegradable polyesters include converting the —OH end group to an ether functionality. In some embodiments, the —OH groups can be replaced by alkyl esters. The end group modification can be performed using methods including Williamson ether synthesis and reaction with diazomethane.

In general, the Williamson ether synthesis is a reaction that converts alcohols (R—OH) into ethers (R—O—R′). The first step in this reaction is forming the conjugate base of the alcohol (called an alkoxide) by reacting the alcohol with sodium metal, sodium hydride, or potassium t-butoxide. In the case with sodium metal, this reaction forms hydrogen gas (H₂) as a byproduct. The alkoxide can then be added to a suitable alkyl halide (typically a primary halide) to form the ether via an SN2 mechanism.

Thus, PLLA-O—R can be formed by first reacting PLLA-OH with sodium metal to form PLLA-O⁻NA⁺. The PLLA-O⁻NA⁺ is then reacted with R′X to form PLLA-O—R′, where X is a halide such as —Cl or —I and where R′ is an alkyl functionality such as —CH₃ or —CH₂CH₃—.

The end group of a biodegradable polyester can also be modified by reaction with diazomethane (CH₂N₂). FIG. 6 depicts a reaction of a linear or star PLLA-OH with diazomethane to form PLLA-O—CH₃. The ethyl ether can also be made by reacting PLLA-OH with triethyloxoniumfluoborate in methylene chloride.

### (5) Urethane Group

In additional embodiments, —OH terminated biodegradable polyesters can be modified to have urethane end groups. The urethane end groups can replace the —OH groups through reaction of the —OH terminated polymer with a mono-isocyanate. Illustrative monoisocyanates include, but are not limited to, methylisocyanate, ethylisocyanate, propylisocyanate, butylisocyanate, pentylisocyanate, hexylisocyanate, decylisocyanate, dodecylisocyanate, tetradecylisocyanate, hexadecylisocyanate, phenylisocyanate, cyclohexylisocyanate, xyleneisocyanate, cumeneisocyanate, and cyclootylisocyanate.

In exemplary embodiments, PLLA-OH can be converted to PLLA-OC(OH)N—R through a reaction with a monoisocyanate, O═C═N—R, as shown in FIG. 7. The reaction can occur at room temperature (20-30° C.) or at an elevated temperature (greater than room temperature) in a suitable solvent and in the presence of a suitable catalyst such as stannous octoate. The stannous octoate catalyst can be that already present in the polymer from its original synthesis. The urethane terminated PLLA polymer can be precipitated from the reaction solution by pouring the solution into a nonsolvent of the polymer.

### (6) Tertiary Amine Group

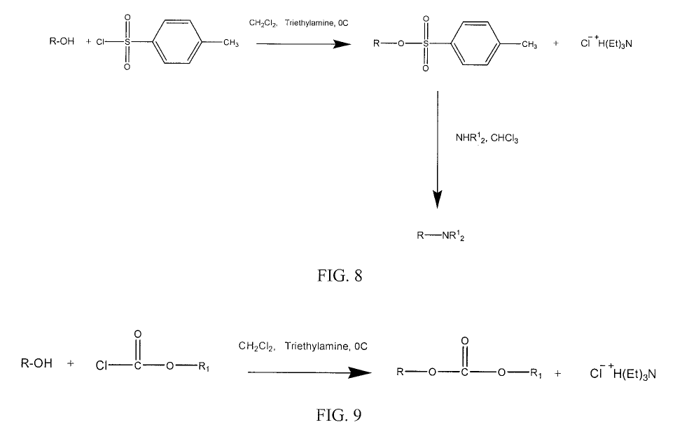

In other embodiments, —OH terminated biodegradable polyesters can be replaced with tertiary amine. A tertiary amine end group can be formed by first converting the —OH to a tosylate, followed by substitution with ammonia, a primary amine, or a secondary amine. FIG. 8 depicts an exemplary scheme of converting a terminal hydroxy group to a teriary amine via a tosylate. In this scheme, R—OH is the hydroxyl terminated biodegradable polyester (e.g., PLLA-OH). And NHR₂¹ is the amine group to be placed at the polymer chain ends. The end group is a primary, secondary, or tertiary amine depending on whether —R₂¹ are both hydrogen, hydrogen and hydrocarbon, or both hydrocarbon.

### (7) Carbonate Group

In further embodiments, —OH terminated biodegradable polyesters can be replaced with a carbonate end group. A carbonate end group can be formed by treating an —OH terminated biodegradable polyester with an alkyl chloroformate. FIG. 9 depicts an exemplary scheme of converting a terminal hydroxy group to a carbonate. In this scheme, R—OH is the hydroxyl terminated biodegradable polyester (e.g., PLLA-OH).

In additional embodiments, an implantable medical device can be fabricated with a polymer material including end group modified polymer and additionally including one or more types of stabilizers mixed or dispersed in the polymer material. Such stabilizers can also further reduce the chemical degradation or decomposition during one or more of the processing steps. Various types of stabilizers can be used including, but not limited to, free radical scavengers, peroxide decomposers, catalyst deactivators, metal scavengers, and water scavengers. In some embodiments, the stabilizing agents can be mixed or dispersed within the polymer during melt processing, such as in an extruder.

One category of stabilizers is free radical scavengers. “Free radicals” refer to atomic or molecular species with unpaired electrons on an otherwise open shell configuration. Free radicals can be formed by thermolysis, photolysis, and oxidation reactions. These unpaired electrons are usually highly reactive, so radicals are likely to take part in chemical reactions, including chain reactions. Free radical scavengers operate through donation of an electron or hydrogen to a free radical, thus removing the free radical from further reaction. The free radical scavenger effectively competes with the polymer for the free radicals, and thus removes the free radicals from the reaction cycle.

Some representative examples of free radical scavengers include, without limitation, oligomeric or polymeric proanthocyanidins, polyphenols, polyphosphates, polyazomethine, high sulfate agar oligomers, chitooligosaccharides obtained by partial chitosan hydrolysis, polyfunctional oligomeric thioethers with sterically hindered phenols, hindered amines such as, without limitation, p-phenylene diamine, trimethyl dihydroquinolones, and alkylated diphenyl amines, substituted phenolic compounds with one or more bulky functional groups (hindered phenols) such as tertiary butyl, arylamines, phosphites, hydroxylamines, and benzofuranones. Also, aromatic amines such as p-phenylenediamine, diphenylamine, and N,N′ disubstituted p-phenylene diamines may be utilized as free radical scavengers. Other examples include, without limitation, butylated hydroxytoluene (“BHT”), butylated hydroxyanisole (“BHA”), L-ascorbate (Vitamin C), Vitamin E, herbal rosemary, sage extracts, glutathione, melatonin, carotenes, resveratrol, propyl gallate, and tertbutylhydroquinone. Examples of some phosphites include di(stearyl)pentaerythritol diphosphite, tris(2,4-di-tert.butyl phenyl)phosphite, dilauryl thiodipropionate and bis(2,4-di-tert.butyl phenyl) pentaerythritol diphosphite. Some examples, without limitation, of hindered phenols include octadecyl-3,5,di-tert.butyl-4-hydroxy cinnamate, tetrakis-methylene-3-(3′,5′-di-tert.butyl-4-hydroxyphenyl)propionate methane and octadecyl-3-(3,5-di-tert.butyl-4-hydroxyphenyl)propionate.

Specific preferred free radical scavengers or antioxidants for biocompatibility include BHT, BHA, trihydroxybutyrophenone, L-ascorbic acid, (Vitamin C), sodium ascorbate, Vitamin E, herbal rosemary, sage extracts, glutathione, melatonin, carotenes, carotenoids, resveratrol, methyl gallate, n-octyl gallate, n-dodecyl gallate, propyl gallate, propyl paraben, luteolin, eriodictyol, astaxanthin, anthocyanins, carnosol, quercetin, catechin, morin, rutin, boldine, tocopherols, hydroxytyrosol, ubiquinol, isoflavones, lycopene, fisetin, ellagic acid, L-DOPA, sinapine, olivetol, dehydrozingerone, curcumin, and tertbutylhydroquinone. Other free radical scavengers, such as various isomers of Vitamin E, may be used, including the four tocopherols and four tocotrienols. The alpha, beta, gamma and delta forms of both the tocopherols and tocotrienols may be used to prevent chemical degradation.

Another category of stabilizers is peroxide decomposers. Peroxide decomposers act by removing an oxidative catalyst present in polymer resins, which is a hydroperoxide. Hydroperoxides readily decompose to create free radicals. Peroxide decomposers react with hydroperoxides to create non-free radical species, and thus help inhibit oxidation. Examples include trivalent phosphorous and divalent sulfur compounds such as sulfites, thiodipropionates and organophosphites. Other examples of peroxide decomposers are esters of β-thiodipropionic acid, such as without limitation, for example the lauryl, stearyl, myristyl or tridecyl ester, and salts of 2-mercaptobenzimidazole, for example the zinc salt, and diphenylthiourea. Among the more stable trivalent phosphorous compounds are dicumylphosphite, tris(2,4 di-tert-butylphenyl)phosphate, and tetrakis(2,4-di-tert-butylphenyl) 4,4′-biphenylenediphosphonite. Also, hydroxylamines are both free-radical scavengers and decompose hydroperoxides.

In the category of peroxide decomposers, preferred compounds for biocompatibility are sulfites, thiodipropionates, β-thiodipropionic acid, such as without limitation, for example the lauryl, stearyl, myristyl or tridecyl ester.Another category of stabilizers is catalyst deactivating agents. These agents reduce the catalytic decomposition of the polymer resulting from residual metal in polymer resins, and may also be referred to as “metal deactivators.” In general these compounds complex with the metal so that the metal can no longer act as a catalyst for the decomposition of hydroperoxides. Non-limiting examples of catalyst-deactivating agents include hindered, alkyl, aryl and phenolic hydrazides, amides of aliphatic and aromatic mono- and dicarboxylic acids, cyclic amides, hydrazones and bishydrazones of aliphatic and aromatic aldehydes, hydrazides of aliphatic and aromatic mono- and dicarboxylic acids, bis-acylated hydrazine derivatives, and heterocyclic compounds. Other compounds include isopropanolamines, phosphate esters, tri-sodium phosphate, tri-potassium phosphate, alkyl or aromatic amines, amides, and alkoxides. A non-limiting example of a specific compound is 1,2-bis(3,5-di-tert-butyl-4-hydroxyhydro cinnamoyl) hydrazine (BNX® MD-1024 from Mayzo or IRGANOX MD 1024 from Ciba-Geigy).

In the category of catalyst deactivating agents, preferred compounds for biocompatibility are amides of aliphatic and aromatic mono- and dicarboxylic acids, cyclic amides, phosphate esters, tri-sodium phosphate, tri-potassium phosphate, L-DOPA, dopamine, 1,4-diaminobutane, 1,5-diaminopentane, and glutathione. An additional category of stabilizers is metal scavengers which includes both chelating agents and cryptands. Cryptands are a “family of synthetic bi- and polycyclic multidenate ligands for a variety of cations.” Cryptands bind cations using both oxygen and nitrogen atoms. Metal chelators and cryptands scavenge and tie up residual metal to prevent the metal from associating with a hydroperoxide which is required to catalyze the depolymerization. Some non-limiting examples of chelating agents are EDTA, DPTA, NTA, and oxalic acid. A non-limiting example of a cryptand is N[CH₂CH₂O CH₂CH₂O CH₂CH₂]₃N.

In the category of metal scavengers, preferred compounds for biocompatibility are ethylene diamine tetraacetic acid (EDTA), porphyrin rings, histidine, malate, phytochelatin, and salts of oxalic acid.

In the category of water scavengers, preferred compounds for biocompatibility are potassium carbonate, carbonates, sodium sulfate, magnesium sulfate, calcium sulfate, calcium chloride, and calcium carbonate. If they are used in nanoparticulate form (<300 nm size) then nanoparticles of aluminosilicates, zeolites, alumina, silica are also possible.

Any type of combination of the above mentioned stabilizers may be used in the various embodiments of the present invention.

In addition to the polymers mentioned above, other representative examples of polymers that may be used to fabricate an implantable medical device according to the embodiments described herein include, but are not limited to, polydioxanone, poly(hydroxy butyrate), poly(butylene succinate), polyesteramide, poly(hydroxy butyrate-co-hydroxyvalerate), poly(butylene succinate adipate), poly(hydroxyl alkanoates), poly(hydroxyl butyrate), poly(hydroxyl hexanoate), and poly(hydroxyl valerate).

For the purposes of the present invention, the following terms and definitions apply:

The “glass transition temperature,” Tg, is the temperature at which the amorphous domains of a polymer change from a brittle vitreous state to a solid deformable or ductile state at atmospheric pressure. In other words, the Tg corresponds to the temperature where the onset of segmental motion in the chains of the polymer occurs. When an amorphous or semicrystalline polymer is exposed to an increasing temperature, the coefficient of expansion and the heat capacity of the polymer both increase as the temperature is raised, indicating increased molecular motion. As the temperature is raised the actual molecular volume in the sample remains constant, and so a higher coefficient of expansion points to an increase in free volume associated with the system and therefore increased freedom for the molecules to move. The increasing heat capacity corresponds to an increase in heat dissipation through movement. Tg of a given polymer can be dependent on the heating rate and can be influenced by the thermal history of the polymer. Furthermore, the chemical structure of the polymer heavily influences the glass transition by affecting mobility.

### EXAMPLES

### Stent Preparation for PLLA with Various Thermally Stable End Groups

PLLAs with different thermally stable end groups are prepared as described above. Then the tubing made from these materials is formed through extrusion in a signal or twin screw extruder at 200° C. The size of the extruded tubing is set at about 0.02″ for inside diameter (ID) and 0.07″ for outside diameter (OD). In case the crystallinity and orientation of the extruded tubing needs to be further increased, the tubing would be expanded in a glass mold at about 90° C. The expanded tubing is cut into a stent by laser machining.

Stent Preparation with PLLA with Various Thermally Stabile End Groups Containing Stabilizing agent

PLLAs with different thermally stable end groups are prepared as discussed above. Then a tube is made from these materials with about 0.2% BHT or vitamin E through extrusion in a twin screw extruder at 200° C. The size of the extruded tubing is set at about 0.02″ for ID and 0.07″ for OD. In case the crystallinity and orientation of the extruded tubing needs to be further increased, the tubing would be expanded in a glass mold at about 90° C. The expanded tubing will be cut into a stent by laser machining.

While particular embodiments of the present invention have been shown and described, it will be obvious to those skilled in the art that changes and modifications can be made without departing from this invention in its broader aspects. Therefore, the appended claims are to encompass within their scope all such changes and modifications as fall within the true spirit and scope of this invention.

What is claimed is:

1. A stent comprising a portion formed of a biodegradable polyester, wherein the polyester has selected end groups that provide greater thermal stability to and reduced chemical degradation the polyester compared to that with hydroxyl end groups.

2. The stent of claim 1, wherein the biodegradable polyester is selected from the group consisting of PLLA, PDLLA, PLLA-co-PGA, PLLA-co-PCL, PLLA-co-PTMC and PLLA-co-PDLLA.

3. The stent of claim 1, further comprising a stabilizing agent mixed within the polymer, the stabilizing agent selected from the group consisting of a free radical scavenger, peroxide decomposer, catalyst deactivator, and metal scavenger.

4. The stent of claim 1, wherein the selected end groups are selected from the group consisting of chlorine, amine, carboxylic acid, acetate, ether, urethane, tertiary amine, and carbonate.

5. The stent of claim 1, wherein the portion comprises a stent scaffolding, coating, or both.

6. A method of fabricating a stent comprising:
processing a biodegradable polyester to form a stent, the processing subjects the polyester to conditions that cause chemical degradation of the biodegradable polyester, wherein the polyester has selected end groups that provide greater thermal stability and reduced chemical degradation to the polyester during the processing than provided by hydroxyl end groups.

7. The method of claim 6, further comprising synthesizing a biodegradable polyester having the selected end groups, the selected end groups selected from the group consisting of chloride, amine, carboxylic acid, acetate, ether, urethane, tertiary amine, and carbonate.

8. The method of claim 6, wherein the conditions comprise a temperature above ambient.

9. The method of claim 6, wherein the processing comprises melt extrusion of the polyester to form a tube.

10. The method of claim 6, wherein the processing comprises radially expanding the tube by blow molding a tube made of the polymer at a temperature above the Tg of the polymer.

11. The method of claim 6, wherein the processing comprises laser machining a tube made from the polyester to form a stent pattern in the tube.

12. The method of claim 6, wherein the processing comprises radiation sterilization of a stent made from the polyester.

13. The method of claim 6, wherein the biodegradable polyester is selected from the group consisting of PLLA, PDLLA, PLLA-co-PGA, PLLA-co-PCL, PLLA-co-PTMC and PLLA-co-PDLLA.

14. The method of claim 6, wherein the selected end groups comprise chloride, amine, carboxylic acid, acetate, ether, urethane, tertiary amine, or carbonate.

15. A method of fabricating a stent comprising:
forming a tube made from a biodegradable polyester using melt extrusion; and
laser machining a stent pattern in the tube to form a stent, wherein the polyester has selected end groups that provide greater thermal stability to the polyester than provided by hydroxyl end groups during the forming and machining steps.

16. The method of claim 15, further comprising radially expanding the tube by blow molding the tube at a temperature above Tg of the polymer prior to laser machining.

17. The method of claim 15, further comprising sterilizing the stent by exposing the stent to radiation.

18. The method of claim 15, wherein the biodegradable polyester is selected from the group consisting of PLLA, PDLLA, PLLA-co-PGA, PLLA-co-PCL, PLLA-co-PTMC, and PLLA-co-PDLLA.

19. The method of claim 15, wherein the selected end groups are selected from the group consisting of chlorine, amine, carboxylic acid, acetate, ether, urethane, tertiary amine, and carbonate.

20. The method of claim 15, wherein a molecular weight decrease in the polyester caused by the melt extrusion, laser machining, or both is less than the corresponding polyester with hydroxyl ends groups.
